(19) United States
(12) Patent Application Publication
(10) Pub. No.: US 2021/0315514 A1
(43) Pub. Date: Oct. 14, 2021

(54) WEARABLE PASSIVE ASSISTING DEVICE

(71) Applicant: MYOSWISS AG, Zurich (CH)

(72) Inventor: Kai SCHMIDT, Zurich (CH)

(73) Assignee: MYOSWISS AG, Zurich (CH)

(21) Appl. No.: 17/272,516

(22) PCT Filed: Sep. 6, 2019
(86) PCT No.: PCT/EP2019/073856

§ 371 Date: Mar. 1, 2021

(30) Foreign Application Priority Data
Sep. 6, 2018 (DE) 10 2018 215 163.6

(51) Int. Cl. A61B 5/00; A61B 5/11; G06F 1/16

(52) U.S. Cl. CPC A61B 5/4561; G06F 1/163; A61B 5/4523; A61B 5/1126

(57) ABSTRACT

A wearable assistive device is suggested comprising a force transmitting interconnection arrangement interconnecting in use a left limb of a user with a right limb of a user in a force transmitting manner; and a deflection arrangement guiding in use the force transmitting member along a path close to the body of a user, the path having a length dependent on the posture of the user; wherein the deflection arrangement comprises at least one elastic element engaging at one end thereof the force transmitting interconnection in between the left limb and the right limb in a manner deflecting the force transmitting interconnection from a straight line by an amount dependent on the excursion of the elastic element.

The present invention relates to a wearable assistive device.

Wearable assistive devices are well known. They help where an exceptionally high load is to be moved by a user or where the movement of the user requires a high effort due to an impairment, for example caused by a disease, accident or aging.

Such wearable assistive devices can be passive or can have active components. Recently, a plurality of active assisting devices having active components such as motors, batteries for the motors, controls, sensors and so forth as well as having passive components such as tendons, ligaments and so forth attached to limb have been suggested to support movement and to exert a force that helps to move the limb as necessary. Active (or: motor powered) systems can be tailored towards optimal assistance by the active element. This holds true for active orthoses such as exoskeletons and exosuits.

Passive systems typically rely either on rigid structures to optimize moment arms in a manner almost independent of the body structure save for cuffs to connect to the human body, and/or they damp certain phases of a movement to release energy in other phases; this however might be disadvantageous due to a pathological gait pattern. Also, some passive systems are added as a stability measure which however requires the user to fight the elements in one direction, which is not beneficial for certain users. Furthermore they are most frequently restricted towards usage within one plane even if routed across multiple joints, e.g. to assistance within the sagittal plane. Even where a known passive system overcomes limitations relying on an anti-phasic movement pattern (phase shifted by 90 degrees) in normal gait, problems will arise in a pathological gait; also, even certain standard arm movements may cause problems.

A soft wearable muscle assisting device having at least a wearable body interface and attached thereon elements of active and passive components adapted to together assist a user in changing or maintaining the position of a first body part articulated to a second body part, the wearable body interface comprising at least two garment portions, the active and passive components comprising at least one actively powered element, the passive components comprising at least a first passive component and a second passive component that both extend between the at least two garment components and comprising elastic material for storing energy provided by the user and/or by the at least one actively powered element wherein the first and the second passive components are arranged on opposite sides of a joint of the body is known from the WO 2018/122106.

An energy expenditure garment made of a variable density fabric having a plurality of zones of differing resistance characteristics by utilizing threads in the fabric of differing resistance characteristics so that the zones are seamless and blend into each other is provided by U.S. Pat. No. 6,047,406.

An antigravity muscle exerciser comprising a garment which when worn by a user is close-fitting and covers at least the torso of said user, the garment comprising separate top and bottom portions or being a one-piece article, and having incorporated therein first portions of flexible material on each side of the torso which extend from the pelvis to beyond a respective shoulder where the portions are contiguous with a second portion of flexible material which extends across the top of the head of the user in a coronal plane, wherein the portions of flexible material in combination have a length and elasticity which creates axial compression between the top of the head and the pelvis is provided in US 2016/0165968.

Furthermore, from WO 2013/060388 A1 a lower back pain training device is known that indicates to the user when a lower back forward flexing movement is made and that provides a possibility to install electronic monitoring of said movement.

From U.S. Pat. No. 4,100,918, a dynamic orthotic knee extension assist device is known that is intended for paraplegic patients and others needing such a device. Cuffs adapted to be secured to a patient's thigh are provided along with mounting means adapted to be secured to a patient's leg below the knee. An elongated elastic member is provided with a means for removably mounting each end thereof to the cuff means at a predetermined tension. Also provided is a means for pivotally securing the elastic means intermediate its ends to the mounting means. A lateral knee brace is rigidly disposed between the cuff and the mounting means. The elastic member provides a flexion moment when the patient is seated. As the patient arises, the flexion moment progressively diminishes and then converts to a progressively increasing extension moment as the patient continues to rise.

A knee joint assistant device using elasticity coefficient change and an optimized method of using walking energy capable of including an elastic tool and a control unit which controls the elasticity coefficient is known from KR 10 2012 0 062 375 A.

From WO 2005/025464 A2, considered to represent the closest prior art, an apparatus for assisting body movement is known including a framework which is connectable with the human body and an elastic force transmission system connected with the framework. The elastic force transmission system stores energy during the first portion of a movement of the human body and releases the stored energy during a second portion of the movement of the human body. The elastic force transmission system includes an elongated force transmission component which is resiliently extendable under the influence of force transmitted through the framework. Although the known apparatus can be used in association with any desired portion of the human body, it is stated to be of particular use in association with one or both legs of the human body. In the prior art document, it is stated that muscles often span multiple joints, which results in energy-saving power transfers when a movement simultaneously requires negative power at one joint and positive power at another joint. It is also suggested to include pulleys in the guide for force transmitting components.

Where wearable assistive devices are used by a patient whose health or physical condition deteriorates progressively, frequent adaption of the support offered by the wearable assistive device might be necessary. The same holds where a patient progressively recovers from surgery, an accident or the like. The wearable assistive device frequently would be easier to accept if the arrangement could be worn for a long time without necessity of recharging batteries other like.

Therefore, a wearable assistive device would be desirable that can be worn for prolonged periods, can be used at least for a plurality of the users as a passive assistive device and which can be easily adjusted to the specific need of a user.

It would be desirable to satisfy at least partially at least some of these needs.

It is an object of the present invention to provide novelties for the industrial application.

The object of the present invention is achieved by the subject matter of the independent claims. Some preferred embodiments are indicated in dependent claims.

Therefore, according to a first general idea of the present invention what is suggested is a wearable assistive device comprising a force transmitting interconnection arrangement interconnecting in use a left limb of a user with a right limb of a user in a force transmitting manner; and a deflection arrangement guiding in use the force transmitting member along a path close to the body of a user, the path having a length dependent on the posture of the user; wherein the deflection arrangement comprises at least one elastic element engaging at one end thereof the force transmitting interconnection in between the left limb and the right limb in a manner deflecting the force transmitting interconnection from a straight line by an amount dependent on the excursion of the elastic element.

Accordingly, forces are not simply transmitted between the limbs on different sides of a user and the limbs move. What is more is that the force is at the same time transmitted in a manner where on the one hand the length of the force transmitting member may change and, additionally, on the other hand, a limb currently to be assisted by transmitting forces may experience a force different from that currently exerted by the movement of the other limb via only the force transmitting member. Instead, a combination of a force transmitted from the other limb and of a force exerted by the elastic element will be experienced at a limb currently assisted. This in turn allows for a much better adaption of assistance to certain movements. Also, the extent of support can be easily changed by exchanging the elastic element against another element that has a different elasticity, by replacing the force transmitting member against another member having a higher or lower elasticity, by changing the length of the elastic element, by changing the anchoring and so forth. This allows optimization of moments exerted on a limb during typical movements to be assisted. Also, despite being a passive device, the wearable assistive device frequently is sufficient for a user where only some muscles or muscle groups need to be assisted while other muscles are capable of still supporting movement in an adequate manner. Here, the wearable assistive device will frequently suffice.

It is noted that a particular useful application is the assistance of hip movements, albeit other limbs can be assisted as well.

In a preferred embodiment, the force transmitting interconnection arrangement comprises an interconnection between the two limbs of the user that is less elastic than the elastic element. In this context, it should be noted that the force transmitting interconnection arrangement may comprise for example wires that are hardly elastic. However, it is also possible to use tendons made from fibers and/or bands having some elasticity; accordingly, the selection of interconnection materials is not restricted to wires and cables. However, it frequently is preferred that the elastic element is more elastic than the interconnection itself. In this way, regarding the additional elongation or shortening of the interconnection arrangement when forces are applied by for example straightening one leg during walking, the majority of extension will take place in the elastic element. This simplifies adaption of the wearable assistive device to the needs of a specific user.

Frequently, it is desirable to provide continuous support to a limb for a cyclic motion. Also, the support frequently should not fall below a certain threshold because otherwise, a user might be unable to support himself/herself, continue with a motion of the user currently carries out; on the other hand, if the support is too high, a training effect for example during convalescence might be impaired. Therefore, it is preferable if the force exerted on the limb does not vary too much over a cyclic motion. For example, the support offered during a cyclic movement may for the entire movement vary by less than 50%.

In a preferred embodiment, the variation of force needed and provided may be even lower, for example only ⅓ or ¼, ⅕ or ⅙ or even only 10% of the force needed during each phase of the cycle. It would be obvious that during a cyclic movement, the force exerted on a given limb to be assisted shall or may vary. However, the degree of support should not vary in an excessive manner. Then, it is also obvious that some users will not need support for the entire movement, for example because only some of the muscles used during a movement are impaired e.g. due to an accident, a neurological disease or aging while other muscles are fully operative. In this case, it is sufficient if the variation of an assistive force exerted on the limb will not vary for more than 50% of the cycle; in case where the impairment of the user is more severe, the variation of support will however be low for at least ⅔ or ¾ or even ⅘ or 9/10 or 100% of the cyclic movement. It should be noted that some variation might be useful so as to have a force behavior that is perceived as more natural, and that such assistive force may even be negative on some phases (that is, the direction of force might reverse). It should also be noted that in most cases, where assistive forces are referred to, reference could as an alternative or in addition be also had to torques.

In a preferred embodiment, the interconnection may comprise a tendon such as a cable made from a plurality of thin wires or may comprise a cord or a ribbon.

It is preferred if the interconnection is guided closely along the wearable assistive device both because in this manner the wearable assistive device becomes less obtrusive and because the snag hazard is reduced.

In a preferred embodiment, the deflection arrangement may comprise at least two pulleys that are spaced apart from each other. In this manner, the interconnection can be easily guided with a smaller radius of curvature without causing extensive friction. For example, one of the pulleys can be arranged at the left shoulder while the second pulley can be arranged on the right shoulder. Note however, that for deflection, a pulley is not an absolute necessity. While a pulley helps reduce friction, other means could be used as well to distribute the pressure exerted by the tendons or other force transmitting interconnection on the human body, using e.g. two metal or plastic plates placed across the left and right shoulder respectively. Where a sufficiently broad force transmitting interconnection element having a low friction coefficient with respect to an underlying material such as a textile garment is used, for example a strong PTFE band, not even plates would be needed and in that case, the deflection arrangement could be considered to be implemented by the broad low friction surface provided across e.g. the shoulders.

It is preferred that the elastic element engages the force transmitting interconnection between the two pulleys or similar parts of a deflection arrangement. In a typical arrangement, the wearable assistive device will be generally symmetrical in that the first of the two pulleys are close to the left limb and the second of the pulleys is close to the right limb. If the elastic element is placed in the middle between these two pulleys, the effect of the elastic element on the interconnection arrangement and hence on the left and right limb will basically be the same.

Furthermore, it should be noted that while advantages may be obtained by a wearable assistive device where a force transmitting element is connected to the limb on only one side of the joint (e.g. dorsal), it is also possible to have an additional second interconnection provided between the left limb of the user and the right limb of the user and connected to the other side of the limb (in the example given ventral), the two interconnections then acting in an antagonistic manner. In such a case, the elastic element can be one single elastic element engaging the first force transmitting interconnection with a first end and engaging the second force transmitting interconnection with a second end thereof. It will be understood that in such a case, pulleys, metal rings or the like could be provided between both ends and the respective force transmitting interconnections so that the elastic element is not fixed to any garment portion but is in a manner floating as self aligning between the two force transmitting interconnections.

Accordingly, where a user needs symmetrical assistance, a symmetrical arrangement wherein the elastic element engages the force transmitting interconnection in the middle between the two pulleys is preferred; it should be noted that variations from an exact symmetry are possible and can even be preferred where the forces to be exerted on the left limb shall differ from those to be exerted on the corresponding right limb. While in certain cases the left and right limbs connected may differ, for example where a person has an amputation and the shank on one side is interconnected to the thigh on the other side or where a limb of the leg needs to be connected to limb of the arm on the opposite side, most frequently, the same limbs would be interconnected, for example with an interconnection running from the left foot to right foot, from left shank to right shank or from left thigh to right thigh.

It should be noted that while generally the wearable assistive device can also be designed to assist movement of the arms, more frequently, the device is to assist movement of the legs and hence the interconnection extends from one leg to the other.

In preferred embodiments, the interconnection will extend beyond more than one joint on at least one limb. For example, a cuff may be placed at the shank and an interconnection tendon can be guided from the cuff placed at the shank via the thigh and the hips across the trunk to the other hip, and downwards to the corresponding thigh and a cuff anchored at the other shank. It should be noted however that other methods of guiding the interconnection tendon and of providing an connecting the tendon to the limb may be chosen. While a cuff placed at the shank right below the knee helps in utilizing the whole length of the thigh segment as a moment arm, this is not necessary and may in some instances be not the preferred embodiment.

It should be noted that guiding a tendon from the left to the right side could either be done via the shoulders with the elastic element engaging the tendon generally parallel to the spine (backbone) and preferably pulling downwards or that the interconnection could be guided across the spine at some point below the shoulders, for example close to the hips. Even though the forces provided by the elastic element need to be sufficient to assist movement, it will be understood that in most cases, these forces do not constitute an additional load to the spine significant to the typical user. Depending on where the tendon or other interconnection is guided across the spine from the left to the right side, the elastic element in a single side setup supporting either flexion or straightening may pull upwards or downwards. It is however to be noted that on certain occasions, a tendency of the anchor point to shift in such a configuration might be observed and that it may be preferable to avoid such tendency. This could be done e.g. by choosing an appropriate (e.g. multi-point) anchoring. In a single side setup supporting either flexion or straightening, the elastic element may pull upwards or downwards. This is true in a single side setup (flexion or extension), although not preferable since the anchor point would try to shift. It should be mentioned that in such a configuration other anchoring points might be more beneficial; and/or that additionally integrating an elastic element into the force transmitting interconnection would be possible as well. In such a case, the interconnection would be somethat elastic and be connected to the anchoring point via a—typically highly elastic—further element.

Also, it is preferred that where the wearable assistive device extends beyond more than one joint on at least one limb, a first part of the interconnection is guided on an anterior path while a second part of the interconnection is guided on a posterior path at a given side. This can be done not only on either the left or the right side of the body, but also simultaneously on both sides, e.g. the left leg and the right leg. Typically, the interconnection on e.g. a left side limb is guided on an anterior path ahead one of the joints and is guided on a posterior path behind the one of the joints.

It should be noted that crossing over from an anterior path to a posterior path may be effected by a variety of measures and in different places, in particular where a knee-brace is used that protects the knee and allows to exert moments in front of the knee. However, while a knee brace can be used, it frequently will be preferred to not use such a rigid structure and different interfaces to the knee can be implemented, although to ease the understanding of the invention, an embodiment making use of a knee brace will be described hereinafter nonetheless.

It should also be noted that passing from the posterior path to the anterior path and vice versa may be done more than once on each side, in particular where the feet are to be assisted as well. While an elastic element might be used that by the forces caused by walking is compressed, usually, it is easier to have an elastic element that is extendable from a position generally at rest. Such an elastic element may be an elastically stretchable ribbon, for example made of textiles or rubber, but could also be a spring, in particular a coil spring, or another suitable pretension system. It should also be noted that current or upcoming technologies such as adjustable dampers that e.g. change their compliance in response to a voltage applied, extendable elements having an extendibility that may be changed by applying a voltage asf. might also be used. Also electromagnetic clutches and/or brakes could be used increasing friction in certain cases or clamping elastic elements previously extended to a specific length. While such elements might consume some electrical energy and could be energized using batteries (unless all energy is e.g. harvested from the motion of the user), they are not active in the sense that a limb of the user itself is moved by an actuator powered electrically. Thus the energy consumption remains very low.

It should be noted that it is possible to use a plurality of such elements simultaneously, for example, by combining in parallel several elements of different elasticity and/or by combining a plurality of elastic elements in series. Combining a plurality of elastic elements simplifies adjusting the support provided to a user. It should be noted that elastic elements combined may not only differ with respect to the spring constant or elasticity module they offer but also with respect to a linearity behavior offering even more possibilities of adjustment. Note that the length of the different elements might also differ, even where for example several rubber bands are used in parallel. In this manner, some elements will only contribute once considerable extension is given. This contributes to adjustability according to a user's needs.

Then, it should be noted that specific advantages may be obtained if some assistance is offered in an antagonistic manner, for example by supporting both flexing and straightening movement of a limb. This may be helpful where the movement of a limb is only impaired for some of the phases, for example because only certain groups of muscles have been damaged. Another reason to provide assistance in an antagonistic manner is that an overall strengthening of the joints might be considered helpful by a number of users, in particular where the user does not feel particularly secure when moving.

It should be noted that further adaptions of the wearable assistive devices are possible other than by altering the elasticity or length of the elastic element and/or by altering the length and/or elasticity of the interconnection. For example, the anchoring points could be changed, for example anchoring the elastic element to the body, the placement of cuffs from which an interconnecting tendon extends and so forth. Thus, adaptation is possible and in some cases needed for example by changing moment arms, springs, pretensions, end stops for motion like STS (sit-to-stand repetitions), providing springs in parallel to adjust the resulting force profiles.

Moving anchor points and/or changing moment arms helps to adjust a torque profile generated by the wearable assistive device on a limb to a profile needed, the resulting torque profile depending inter alia on the chosen moment arm at the lowest point of for example the thigh segment and relative to the hip joint. Additional structures can be used anteriorly or posteriorly to adapt the moment arm with respect to a joint. This may generate a force bias in the one direction, in particular the direction with the longer lever arm, and provides an additional means of adjusting the forces and resulting torque profiles. It should also be noted that forces in opposing directions may add stability in the same way co-contracting muscles do.

By changing moment arms for example when using a knee -brace arrangement, certain biases can be generated that are potentially tailored better towards distinct gait deficiencies. As an example when using a knee -brace arrangement, additional elastic rubber bands could be provided at different anchoring points, altering the moments generated. Also, the wearable assistive device can provide support in an antagonistic manner, that is, it is possible that at least one elastic element with a first end thereof engages the first force transmitting interconnection while the second force transmitting interconnection is engaged with the second end of the elastic element. Again, the elastic element is not restricted to consist only of one single elastic end.

The present invention will now be described with reference to the Figures. In the drawings:

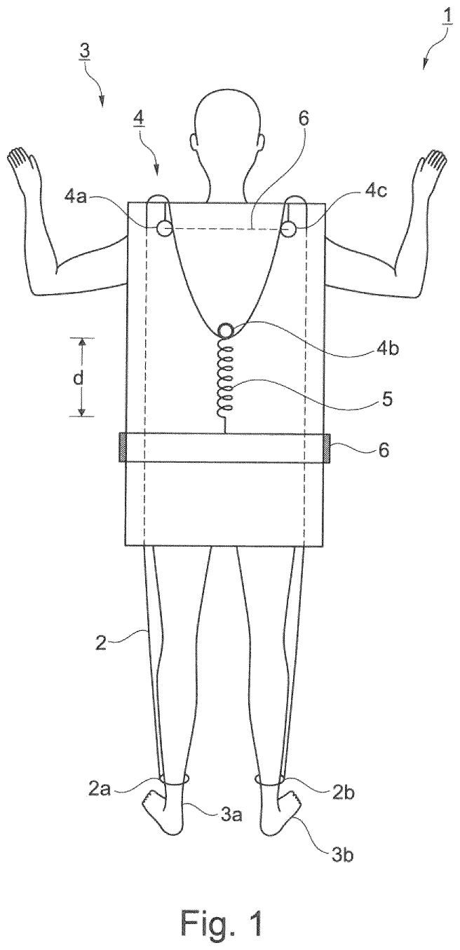

FIG. 1 shows the schematics of the wearable assistive device as worn by a user, showing the back of the user;

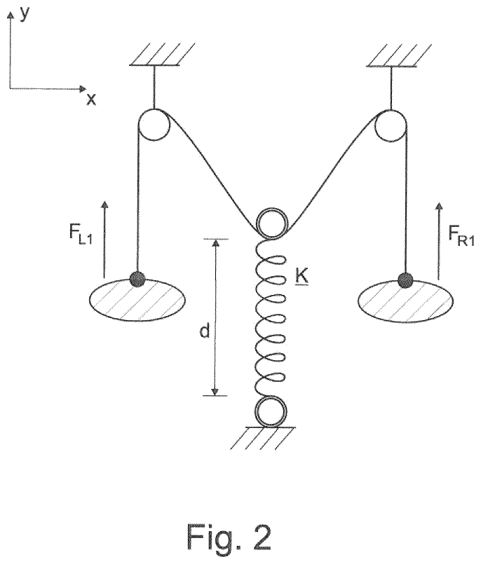

FIG. 2 shows a model of forces exerted by the wearable assistive device shown in FIG. 1 in rest position;

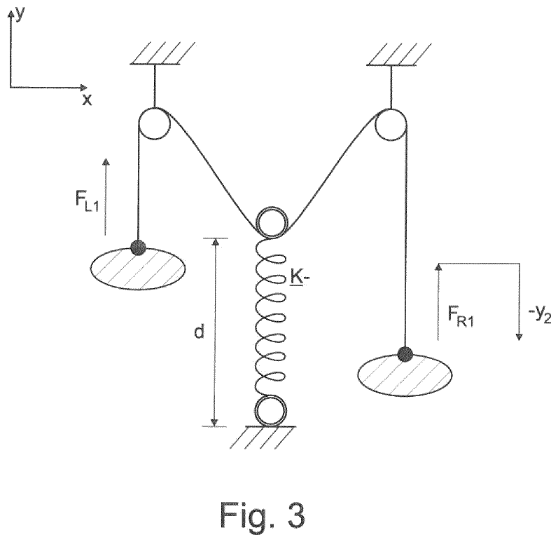

FIG. 3 shows the same as in FIG. 2 during movement;

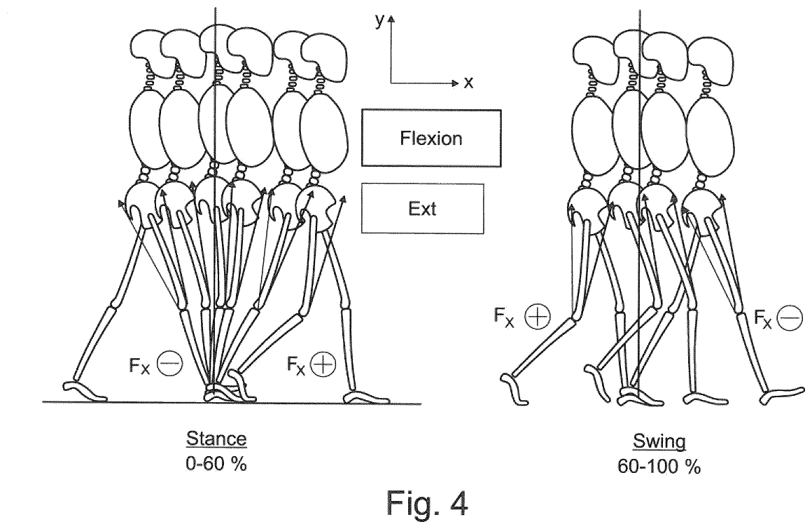

FIG. 4 shows an illustration of the movement of a user showing different points during a stance phase and a swing phase;

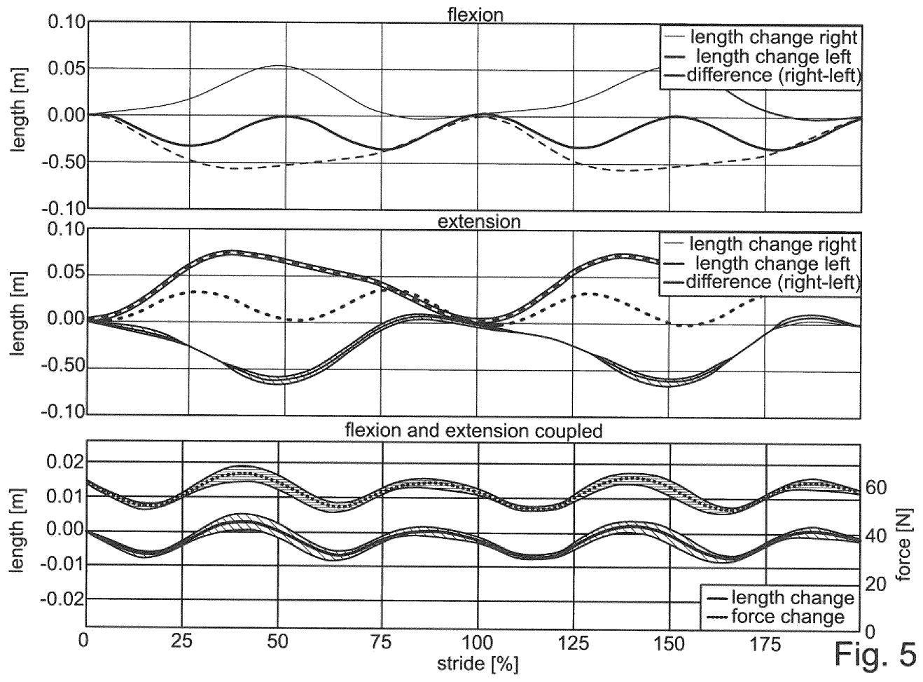

FIG. 5 shows the differences in change of length of an elastic element when coupled to different interconnections between left and right legs; with
the upper curve relating to an interconnection coupling the legs in a flexing movement,
the middle curve relating to an interconnection coupling the legs in an extending movement,
the lower curve relating to an interconnection coupling the legs in both a flexing and an extending movement.

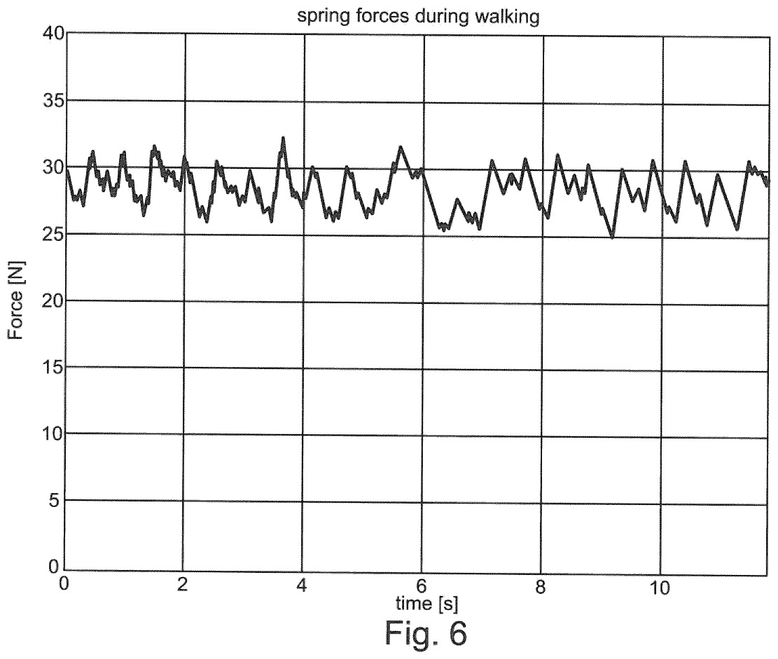

FIG. 6 shows forces during walking in an elastic element in a practical embodiment of the invention, more particular while walking with a system in which both legs are connected to an elastic element in both extension and flexion direction (hip, unimpaired);

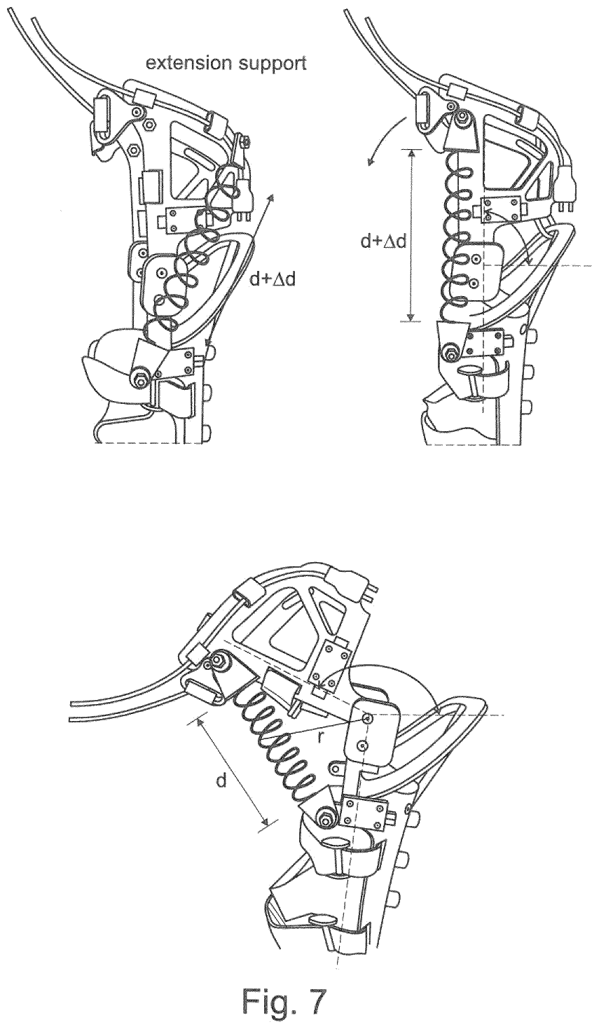

FIG. 7 shows a knee brace with elastic elements attached to different anchor points and showing the knee brace straight and flexed respectively;

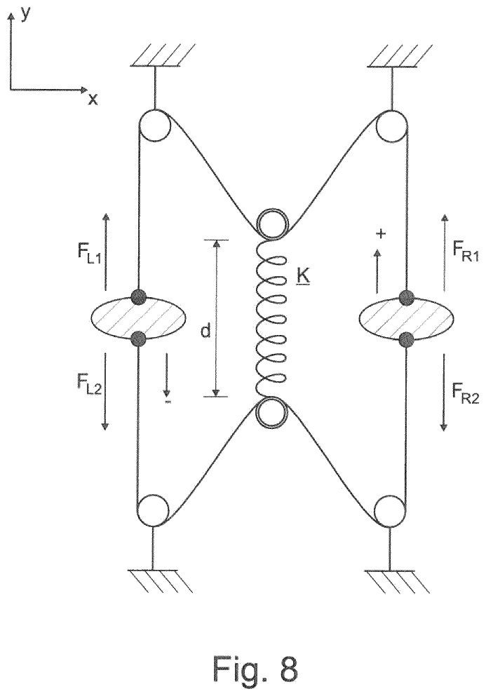

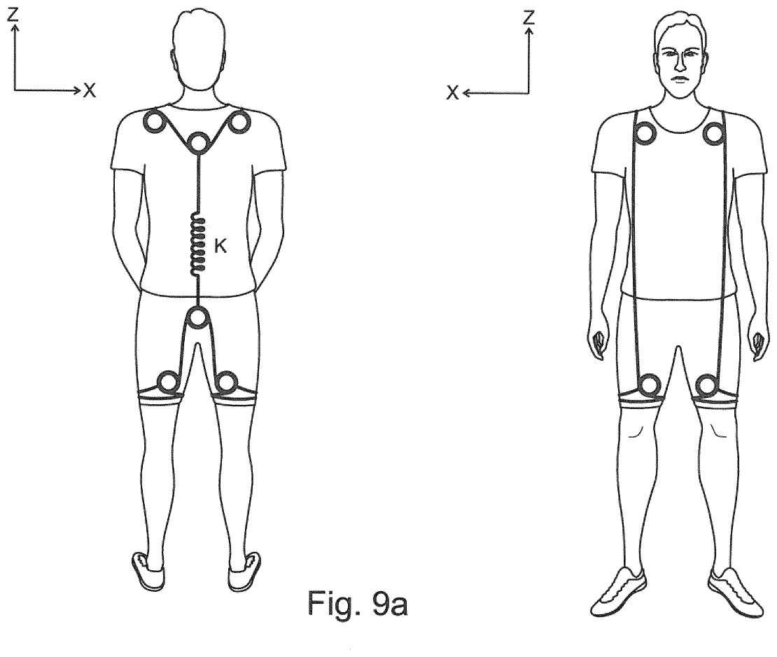

FIG. 8 shows a model assistive device providing antagonistic forces;

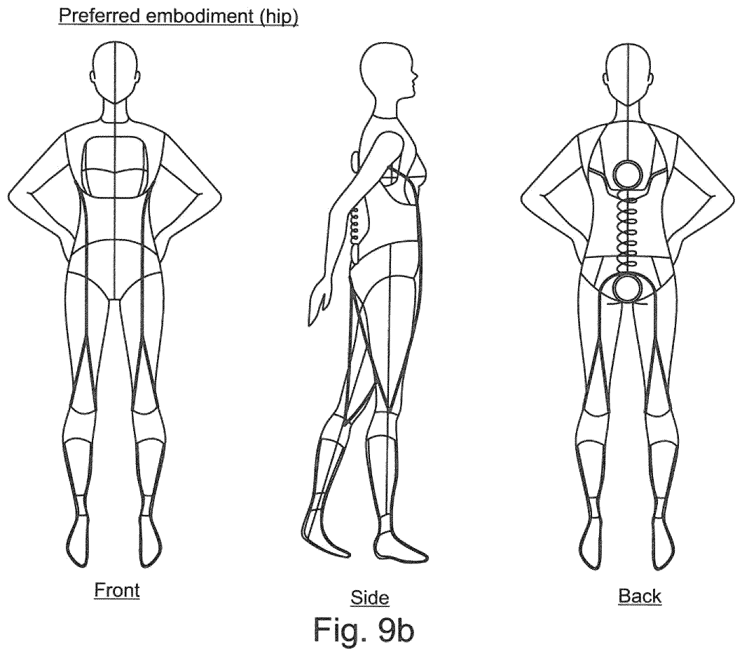

FIG. 9a shows an example for a passive assistive device using the elastic element both for a flexing and a extension movement;

FIG. 9b shows a schematic drawing of an embodiment as shown in FIG. 9a;

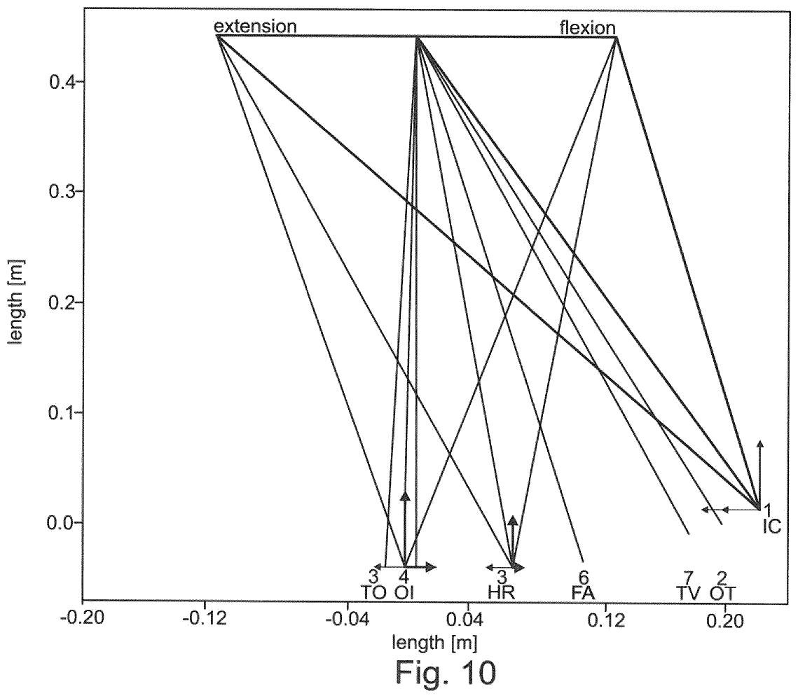

FIG. 10 shows the forces acting in flexion and in extension direction at the endpoint of the thigh segment, with unimpaired, normal walking with
1 IC indicating “Initial contact”,
2 OT indicating “Opposite toe off”,
3 HR indicating “heel rise”,
4 OI indicating “Opposite initial contact”,
5 TO indicating “Toe off”,
6 FA indicating “Feet adjacent”,
7 TV indicating “tibia vertical”;

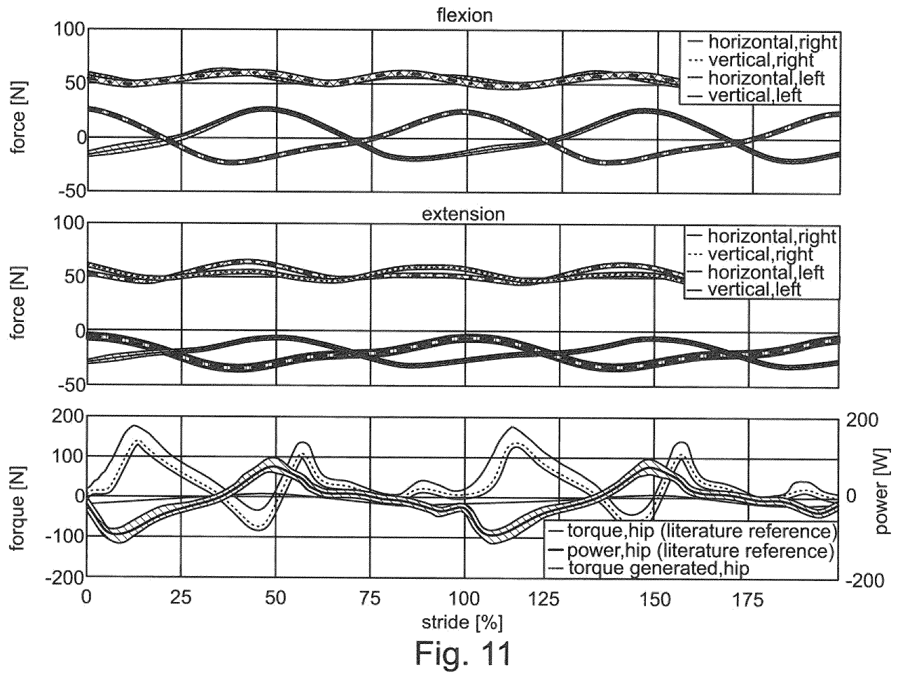

FIG. 11 shows forces and torques resulting because of an anti-phasic interconnection at the hip for flexion and extension;

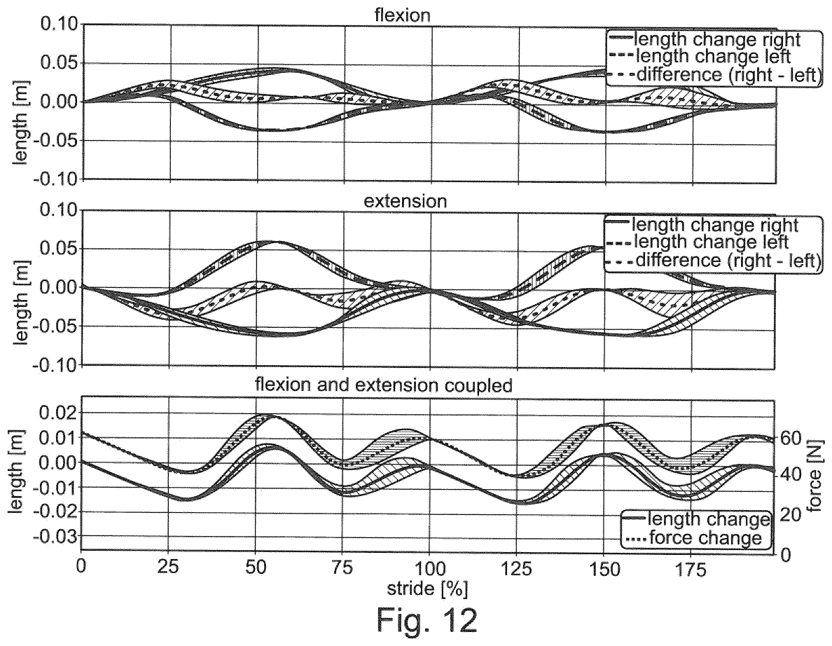

FIG. 12 results achieved with the assistive device of the present invention for a user having an abnormal gait due to extensive hip circumflexion;

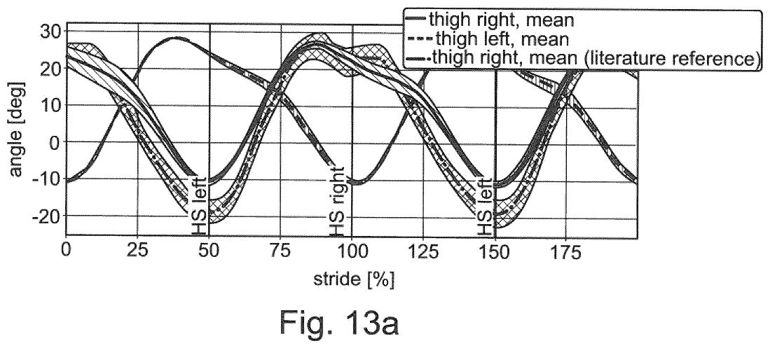

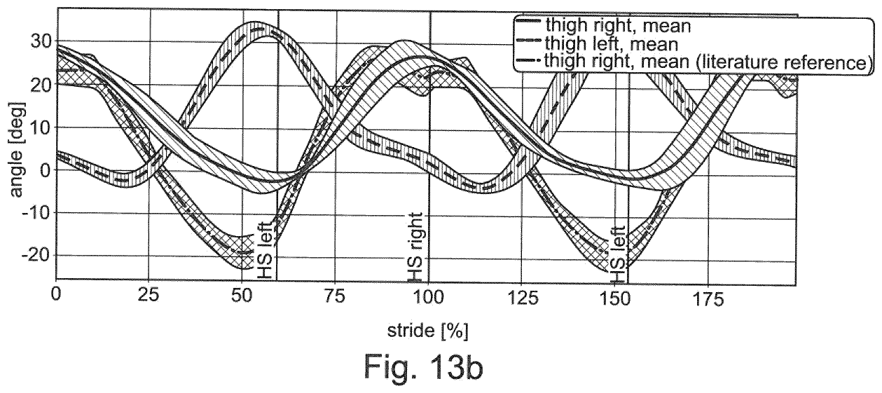

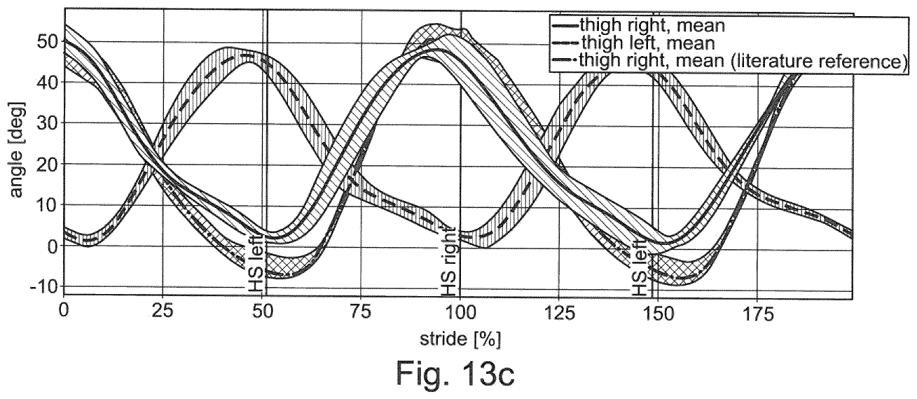

FIG. 13a-c the thigh angle for a cycle of normal walking, namely

FIG. 13a the thigh angle for a cycle of walking pathological due to MD—muscle dystrophy;

FIG. 13b the thigh angle for a cycle of walking upstairs in an unimpaired manner;

FIG. 13c knee angle normal walking;

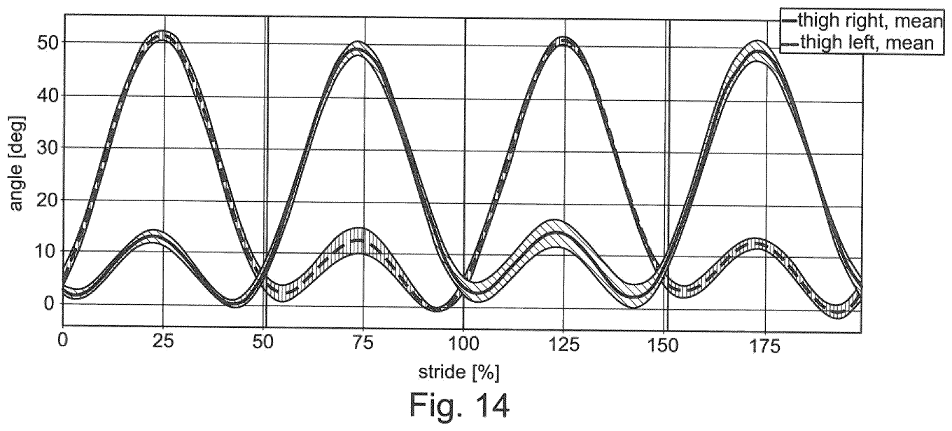

FIG. 14 example of anti-phasic movement that does not allow for equal cable release of force transmitting components is a knee joint where a pattern as shown in FIG. 14 is obtained

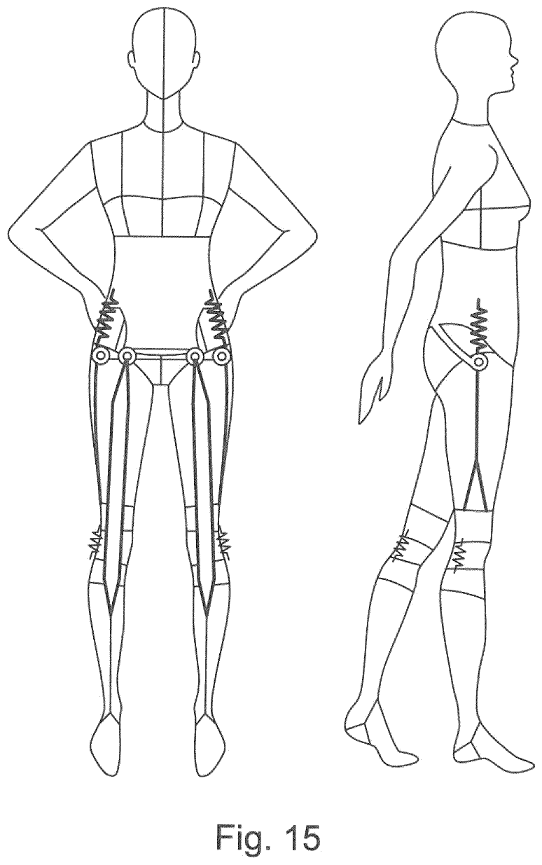

FIG. 15 a connection of the knee to the IT band;

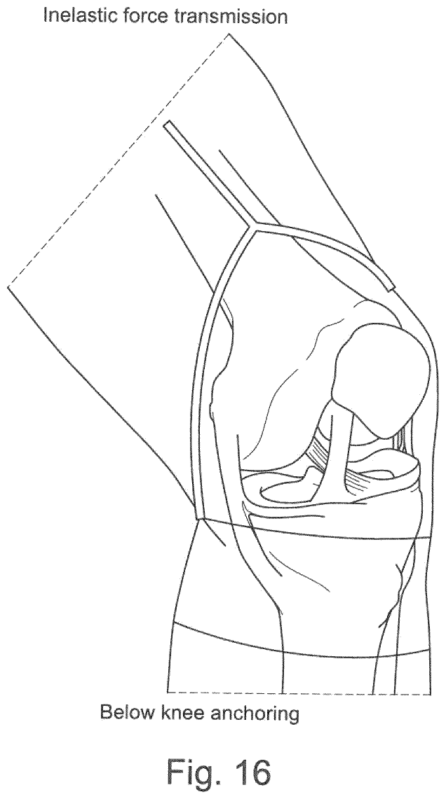

FIG. 16 an attachment of a cuff below the knee.

According to FIG. 1, a wearable assistive device 1 comprising a force transmitting interconnection arrangement 2 interconnecting in use a left limb 3a of a user 3 with a right limb 3b of a user in a force transmitting manner and a deflection arrangement 4 guiding in use the force transmitting member 2 along a path close to the body of a user, the path having a length dependent on the posture of a user, wherein the deflection arrangement 4 comprises an elastic element 5 engaging at one end thereof the force transmitting interconnection in between the left and the right limb in a manner deflecting the force transmitting interconnection from a straight line 6 by an amount dependent on the excursion d of the elastic element 5.

Note that in this embodiment, the elastic element is supportive for a given movement of a given limb in only one direction (either flexion OR extension). This is not because having the elastic element work in only either flexion OR extension is preferred, but because the general ideas are easier to be understood for this embodiment. However, using the elastic element in a manner supportive for a given movement of a given limb in only one direction usually works best in a single segment use cases, e.g. where movement of only the hip or only the shoulder is to be assisted, and/or where the movement itself shows symmetry e.g. hip. However, using the elastic element in a manner supportive for a given movement of a given limb in only one direction usually is not the preferred embodiment for a multi-segment setup, or where a joint is in the middle of a kinematic chain, (that is the joint is distal to the trunk, or the joint is distal to the attachment; typically, this holds for the knee and the elbow).

Also, it should be noted that when using the elastic element in a manner supportive for a given movement of a given limb in more than one direction, anti-phasic extension/flexion movement will further improve the application of a constant force without hindering any movement or compensation. Then, using the elastic element in a manner supportive for a given movement of a given limb in more than one direction provides stability and allows free movement and does not rely on damping phases in the movement. Even where anti-phasic movements are not fully equal, e.g. at the knee, they can be still be used to help support the passive structures within the human body (e.g. IT band). Also, even where an anti-phasic movement exists but does not allow for equal cable release of force transmitting components, simple measures such as additional rubber bands can be used to tailor certain supportive torque profiles e.g. knee. An example of anti-phasic movement that does not allow for equal cable release of force transmitting components is a knee joint where a pattern as shown in FIG. 14 is obtained.

Note that where these (knee) movements that are not equally anti-phasic are to be used to support the passive structures within the human body (e.g. IT band), a routing outside of the plane of force application is beneficial. An example of such a connection is shown in FIG. 15. FIG. 15 shows a connection of the knee to the IT band; it is possible to tailor the resulting torque profiles using such a structure despite the lack of a rigid brace.

Thus, most of the times, using the elastic element in a manner supportive for a given movement of a given limb in more than one direction is preferred.

The wearable assistive device 1 in the embodiment shown in FIG. 1 assists a user 3 having in the present case a general weakness of muscles of both legs. It is to be noted however that even where a user only has one leg being so weak that it needs assistance (that is even where the user has one healthy leg), the device will still provide valuable assistance as well as in that case the healthy or better leg may help the user compensate the weakness of the weaker leg.

The interconnection 2 in the embodiment shown in FIG. 1 comprises a tendon made out of basically non-extendable cable. Non-extendable in this context means that the extension under forces exerted by the user during normal use is less than 5% of the extension of elastic element 5 under the same force. Note that such a low extendibility is chosen for the embodiment as the arrangement is easier to understand but that in a practical implementations the extension of the interconnection might have a higher percentage.

The tendon 2 ends on the left limb 3a at a cuff 2a attached around the shank of the user 3. (This could be done directly below the knee as shown in FIG. 16. In the manner shown in FIG. 16, the thigh can be used to provide a rather longest moment arm in a manner not adversely affecting the knee.)

The tendon 2 in the embodiment shown can be guided to a brace of the knee such as the brace shown in FIG. 7. Note however, that providing a knee brace having rigid parts is not necessary for many users and that an overall soft structure is preferred frequently. In this context, it will be understood that with respect to the knee, a routing outside the plane of force application is beneficial, as it allows movement without restriction, does not rely on damping phases and might help the person to return into a more normal symmetric gait.

As can be seen, the tendon 2 branches at the knee into two parts, one part being guided on the left side of the left knee, the other part being guided on the right side of the left knee. Behind the knee, the tendon parts join again.

Returning to FIG. 1: Above the knee, the tendon 2 is guided across the hip joint, guided along the trunk of the user on the front side to the left shoulder, is guided across the shoulder and passed along a pulley (or another low-friction metal element such as a metal ring) which is anchored at the shoulder and constitutes part of the deflection arrangement 4.

The tendon 2 is guided to a further pulley 4b arranged approximately at the position of the spine but significantly below the shoulders. The axle of the pulley is held in engagement with the end of spring 5 which in turn at its opposite end is anchored to a belt 6. In the embodiment shown, the arrangement is symmetrically on the right side. Note however that in case forces start to act in the system, the arrangement need not remain symmetrical but will self-align in an asymmetrical manner corresponding to asymmetrically acting forces.

While not shown in the schematics of FIG. 1, part of the tendon 2 may pass from an anterior to a posterior path for example when passing the hip joint. The tendon 2 may be guided in loops attached to pieces of garment worn by a user or in a slack sheath as needed for comfort and in order to ensure proper placement of the tendon so as to provide the forces and flexing or bending moments as needed. The elements depicted such as the pulleys, tendons, cuffs, belt and the like are arranged on a single piece of garment or on a plurality of pieces of garment that can be worn by the user, preferably under normal clothing.

The belt 6 is arranged such that the forces acting on the respective end of the spring attached thereto will be distributed across the body of the user 3 in a manner not inflicting pain or discomfort to the user to the best degree possible. It should be noted that while reference is made to pulleys for deflecting the tendon 2, other arrangements could be used such as metal rings providing low friction between tendon-like cables and the ring itself, textile groups and so forth. Also, it will be understood that in contrast to a providing a cable as tendon 2, the interconnection arrangement might also comprise a rope made of flexible elements and materials.

Simplifying the schematic FIG. 1 further, it can be seen that in a rest position the elastic element 5 exerts forces on the tendon 2 pulling symmetrically at both cuffs 2a 2b arranged around the shanks of a user.

The force acting on the cuffs depends on the elongation of the elastic element 5. Now, if the user moves, for example because he/she is walking, the overall path length between the left and the right cuff will change. As an example of the movement of a human body, vis-a-vis a stance phase where the foot of the respective leg remains on the ground, during the swing phase where the foot is moving away from ground, the length needs to be changed. This remains valid when considering simultaneous real movements at both sides which generally will not be perfectly anti-phasic. Furthermore, it should be realized that the stiffness of the human body may have an influence on the overall length as well.

As the cable or tendon 2 itself is assumed to be inelastic in the embodiment shown, such change in length can only be accommodated for by extending spring 5 to a larger or to a smaller degree, effectively changing the angle between pulleys 4a, 4b and 4c as shown in FIG. 1.

Thus, movement is associated with a change of length of the spring. Given the elasticity of the spring 5, this will alter the energy stored within the spring so that during the movement, energy is stored in the elastic element or retrieved therefrom. It should then be noted that the energy stored in or released from the elastic element will depend on the elasticity of the element and hence is adjustable. Where a movement is perfectly anti-phasic, it may be desired to store as little additional energy other than one stored by the pretension as possible.

Furthermore, it will be understood that the extension of the interconnection needed during motion will depend on the way it is guided along the human body. This is shown in FIG. 5 for two different ways of guiding the tendon along the body, namely in front of the hip on the one hand and on the rear side of the hip on the other hand. All other points being equal, thus, by guiding the tendon appropriately, the assistance provided by the wearable assistive devices during movement can be adapted as well.

For the sake of completeness, it should be noted that variations of the curve shown in FIG. 5 will occur from user to user and for different activities. This can easily be seen from the difference of the thigh angle shown in FIG. 13a-c for a cycle of normal walking, for a cycle of walking pathological due to MD—muscle dystrophy and for a cycle of walking upstairs in an unimpaired manner.

Using a practical implementation of a device as schematically shown in FIG. 1 shows that a strongly periodic behavior can be observed during normal walking. From the above description it would be easily understood that the overall force will depend on the strength of the elastic element of the pretension used.

FIG. 7 shows that in addition to the forces that stem from the combination of the interconnection and the elastic element, additional forces can be obtained where a brace in particular for the knee is used. In frequent cases, a brace for the knee is necessary as the knee is frequently damaged during accidents so that additional support is needed at least initially, despite the fact that a knee brace having rigid parts certainly is not preferred over soft structures. In addition to the support provided by the knee brace, the knee brace can also be used as part or together with the wearable assistive device for guiding the cable e.g. in front of the knee so that a proper moment can be exerted on the knee joint for flexing or bending. Also, it may be preferred in case a knee brace is used at all that a plurality of anchor points are provided at a knee brace so that different anchor points for an elastic element assisting in bending or flexing the knees can be selected. By selecting the correct anchor points, the moments exerted on the knee during movement can be adjusted.

The wearable assistive device schematically shown in FIG. 1 is used as follows:

First of all, a user puts on a piece of clothing that embodies the wearable assistive device. This piece of clothing could be worn above the users normal clothing; for users having severe physical difficulties to dress and undress themselves, this allows to first help dress and undress them in a standard manner and to thereafter help the user put on the wearable assistive device, reducing the need to adapt or learn certain routines for dressing; however, in a typical case, it is possible and typically will be preferred if the wearable assistive device is worn under normal clothing. As the wearable assistive device can be made rather small, the clothing worn above the wearable assistive device needs to be only slightly oversized if any. Note that it might be preferred that the wearable assistive device is worn above the underwear of the user. This might be preferred for hygienic reasons and as it is possible to put on the wearable assistive device without causing creases in the underwear worn below, so that the wearable assistive device may be sufficiently tight for providing assistance without causing discomfort.

With this, the proper elastic elements having elasticity and length so as to provide useful assistance to a given user are provided across the knee brace and at his back respectively.

The cuffs are then applied to the shanks and the user gets up and starts walking. When walking, the prearranged tendon that has been anchored at the left and right shank respectably exerts a force on the element, extending the element. The combined force from the extended element and the other limb is transmitted via the tendon 2 to the cuff of the opposite limb.

The user thus experiences a force on both limbs.

While moving, the length of a tendon changes according to the bending of joints and so forth and depending on posture. At the same time, energy is stored in or retrieved from the elastic elements a force is exerted on the limbs.

However, for example when walking, one of the legs will usually be in a stance phase and during such phase, extension assisting forces are usually required to move the body weight over the standing leg, whereas flexion assisting forces are needed when swinging the leg forward. In particular, the other leg will at least during some time of the stance phase of the first leg be lifted from ground and a useful moment and force will be exerted on this “free” limb, using the assistive device thus contributing to the movement thereof. Therefore, despite the device being a passive device, a force can be exerted on a limb, energy can be provided from the energy stored in the elastic element and a movement of the user can thus be assisted.

It should be noted that it frequently is helpful to support a user in an antagonistic manner, that is providing forces that simultaneously act on a joint in a flexing and straightening manner. This holds for the present device as well. It should be noted that in certain cases a net force either bending or flexing at least during some stages of the movement will be exerted. Also, note that frequently forces acting vertically at a given joint generate torques at the joint that over time act in opposite directions, while forces acting horizontally frequently will usually show phases where they act in the same direction creating a flexion or extension torque.

Now, it is possible to assist a user in an antagonistic manner using a wearable assistive device of the present invention as well. A first possibility would be to provide two tendons arranged on at the least one joint such that at this joint, an antagonistic behavior is observed, that is for example so that one of the tendons supports a flexing movement while the other tendon supports the extension movement.

While assistance could be completely independent, for example as two different elastic elements could be provided, another possibility is shown with respect to FIG. 8 and FIG. 9.

Here, only one elastic element (or a group of elastic elements in parallel and all in series, but all attached at the same points) is used for deflecting both a first tendon and a second tendon providing a behavior antagonistic to the first tendon. The respective tendons may be guided along different paths of the body but anchored at the same limbs. It will be understood that these different paths may be such that an antagonistic behavior is obtained, for example using one path anterior and another path posterior. As schematically shown, in such a case two pairs of pulleys or other similar arrangements such as low-friction metal rings, namely one pair for each tendon, cable or interconnection, are used and by attaching the elastic element such that the first end thereof engages with the first tendon while of the second end of the elastic element engages with the second tendon. Thus, the elastic element is floating between the interconnection elements and will self-align. In this manner, a tighter coupling of the antagonistic arrangements is achieved, and also the number of parts and/or the weight of the wearable assistive device is reduced. It should be noted that while FIG. 8 is a schematic similar to that in FIG. 2, a practical implementation will again comprise several pieces of garment made from sufficiently tough, light textiles and so forth that preferably can be worn above conventional clothing.

It will be easily understood that the arrangement of FIG. 8 and FIG. 9 improves the assistance to a user in particular where the gait results in highly anti-phasic movements of the left and right legs. This can be seen in particular from FIG. 5 which shows the differences in change of length of an elastic element when coupled to different interconnections between left and right legs with the upper curve relating to an interconnection coupling the legs in a flexing movement, the middle curve relating to an interconnection coupling the legs in an extending movement and the lower curve relating to an interconnection coupling the legs in both a flexing and an extending movement.

It can be concluded that given the anti-phasic movements, the length change needed when connecting to the thigh of both the left and the right leg in either flexion (anterior connection) or extension (posterior connection) is already much less than the required travel (length) per leg. Nonetheless, as these difference in extension and flexion direction are anti-phasic, an overall length change needed can still be significantly reduced compared to the travel of either posterior or anterior connection by coupling the flexion and the extension interconnection as shown in FIGS. 8 and 9. It will be understood that while FIG. 9a shows parts of an assistive suit according to the invention using two interconnections for assisting flexion and tensioning, respectively, FIG. 9b shows another schematic view of such an arrangement having an elastic element that engages with both interconnections in a floating manner. By using end stops, both arrangements are able to support sitting transitions as well. Because both legs move synchronously, the spring in the back tensions accordingly if the deflection arrangement 4 (FIG. 1) is preventing more ribbon or tendon to move towards the posterior side of the body.

Movements that are intrinsically anti-phasic, such as walking or stair climbing, result in the lowermost plot for which both the flexion and the extension interconnection are coupled through the same elastic element; in the example shown, the force setpoint was 60N and it can be seen that the forces fluctuate only very slightly around that point (+−10%). Such slight variation is most frequently extremely helpful for a user.

These results are made possible by the disclosed arrangement used instead of a “direct” spring connection that would only span a joint with one sole passive element and only directly, as in such a case little pretension would be achieved, as can be estimated for a standard case where a rubber band 4 cm wide having rest length 10 cm and 4 cm elongation would be used.

Regarding forces acting in flexion and extension, reference is made to FIG. 10 showing the length behavior due to forces acting in flexion and extension direction at the endpoint of the thigh segment in unimpaired, normal walking with 1 IC indicating “Initial contact”, 2 OT indicating “Opposite toe off”, 3 HR indicating “heel rise”, 4 OI indicating “Opposite initial contact” 5 TO indicating “Toe off”, 6 FA indicating “Feet adjacent”, 7 TV “indicating tibia vertical.” However, forces are depicted only Initial contact”, “heel rise”, and “Opposite initial contact”. It can be seen that the vertical forces stay almost constant which helps with stability, whereas forces acting horizontally are generating flexion and extension torque biases around the joint.

If forces and torques provided by the wearable assistive device of the present invention are plotted for a full cyclic motion such as in FIG. 11, showing forces and torques resulting because of an anti-phasic interconnection at the hip for flexion and extension. This sort of interconnection is obtained using a wearable assistive device as shown in FIG. 9, where two interconnections are provided between the left and the right limbs, namely one for flexion and one for extension. It can be seen that the horizontal forces are providing stability, whereas the horizontal forces result in moments that support flexion and extension. It should be noted that the resulting moment/torque curves plotted compare well to literature and show that the delivered assistance is meaningful (20% in extension and 10% in flexion direction).

Similar results can be achieved in an abnormal gait pattern. As an example, FIG. 12 shows results achieved with the assistive device of the present invention for a user having an abnormal gait due to extensive hip circumflexion—as can be seen, even though the deviations in force are slightly bigger, using the same elastic element for both flexion and extension movements yields results much better than by other attachment of the elastic element.

It will be understood that the embodiment of FIG. 9 is of particular use when assisting hip movements. Hence, it is preferred to use the device of the invention as a wearable hip movement assistive device.

Given that the arrangement disclosed needs no electro-actuator and control, high capacity batteries and so forth, the assistive device can be produced in a cost-effective manner and will be particularly light weight and will not be discomfortable to a user even during prolonged use. Here, it is helpful that for most users, no large rigid structures such as knee braces are needed, even where small metal elements such as guiding rings or small pulleys may be used. Also, the assistive device will be easy to clean, allowing in particular to easily launder or dry clean the entire arrangement or at least the textile portions thereof which can very easily be separated from the few parts such as metal springs or pulleys that should preferably not be laundered.

Given the results disclosed, applicant also considers that while currently no claim is directed to such assistive device, a wearable assistive device having a first force transmitting interconnection arrangement interconnecting in use a left limb of a user with a right limb of a user in a force transmitting manner; and having a second force transmitting interconnection arrangement interconnecting in use a left limb of a user with a right limb of a user in a force transmitting manner; wherein the first force transmitting interconnection arrangement assists in flexion of at least one joint of at least one limb while the second force transmitting interconnection arrangement assists in tensioning (straightening) of that very joint, and wherein the first and second force transmitting interconnection arrangements are coupled to each other in a manner transmitting forces from one interconnection arrangement to the other and wherein at least one, preferably both of the first and second force transmitting interconnection arrangements have or are made up of elastic elements allowing an elongation when forces during use are exerted, is considered inventive as well. Applicant reserves the inter alia right to claim such additional assistive devices in divisional applications, in particular but not only as knee assistive devices. Note that at the time of application, it is considered that such additional devices might be claimed even though no deflection arrangement is provided having at least one elastic element engaging at one end thereof the force transmitting interconnection in between the left limb and the right limb in a manner deflecting the force transmitting interconnection from a straight line by an amount dependent on the excursion of the elastic element.

1. A wearable assistive device comprising:
a first force transmitting interconnection arrangement, wherein the first force transmitting interconnection arrangement is configured to interconnect a left limb of a user with a right limb of a user in a force transmitting manner; and
a deflection arrangement, wherein the deflection arrangement is configured to guide the first force transmitting interconnection arrangement along a path close to the body of the user, the path having a length dependent on the posture of the user;
wherein the deflection arrangement comprises at least one elastic element, wherein a first end of the at least one elastic element is configured to engage the first force transmitting interconnection arrangement so that it is positioned between the left limb and the right limb of the user in a manner deflecting the first force transmitting interconnection arrangement from a straight line by an amount dependent on an excursion of the at least one elastic element.

2. The wearable assistive device according to claim 1, wherein the first force transmitting interconnection arrangement comprises an interconnection that is less elastic than the at least one elastic element.

3. The wearable assistive device according to claim 1, wherein the elasticities of the first force transmitting interconnection arrangement and of the at least one elastic element are selected such that for a cyclic motion the force exerted varies by less than 50% for at least 50% of the cycle.

4. The wearable assistive device according to claim 1, wherein the first force transmitting interconnection arrangement comprises a tendon or a web or a ribbon.

5. The wearable assistive device according to claim 1, wherein the deflection arrangement comprises at least two pulleys or low-friction metal elements spaced apart.

6. The wearable assistive device according to claim 5, wherein the at least one elastic element engages the first force transmitting interconnection arrangement between the two pulleys or low-friction metal elements.

7. The wearable assistive device according to claim 1, wherein the first force transmitting interconnection arrangement is configured to interconnect the left limb and the right limb of the user, wherein the left limb and the right limb are the same limbs.

8. The wearable assistive device according to claim 1, wherein the the left limb and the right limb are part of the legs of the user.

9. The wearable assistive device according to claim 1, wherein the first force transmitting interconnection arrangement extends beyond more than one joint on at least one of the left limb or the right limb, and wherein a first part of the first force transmitting interconnection arrangement is guided on an anterior path while a second part of the first force transmitting interconnection arrangement is guided on a posterior path on said limb.

10. The wearable assistive device according to claim 9, wherein the first force transmitting interconnection arrangement is guided on an anterior path ahead of the one joint and is guided on a posterior path behind the one joint.

11. The wearable assistive device according to claim 1 wherein the elastic element is extendible.

12. The wearable assistive device according to claim 1, wherein the elastic element comprises one of a spring or an elastically stretchable ribbon.

13. The wearable assistive device according to claim 1 wherein a second end of the elastic element is fixedly anchored to the wearable assistive device.

14. The wearable assistive device according to claim 1 further comprising a second force transmitting interconnection arrangement, wherein the second force transmitting interconnection arrangement is configured to be positioned between the left limb and the right limb of the user, the first force transmitting interconnection arrangement and the second force transmitting interconnection arrangement acting in an antagonistic manner.

15. The wearable assistive device according to claim 14, wherein the at least one elastic element comprises a first end and second end, wherein the first end is configured to engage the first force transmitting interconnection arrangement and the second end is configured to engage the second force transmitting interconnection arrangement such that the at least one elastic element is floating between the first force transmitting interconnection arrangement and the second force transmitting interconnection arrangement.

16. The wearable assistive device according to claim 4, wherein the interconnection comprises a tendon, and wherein the tendon is guided in part within slack sheaths or in textile loops.

17. The wearable assistive device according to claim 15, wherein the limbs are the feet and/or the shanks and/or the hips.

18. The wearable assistive device according to claim 1, wherein the elasticities of the first force transmitting interconnection arrangement and of the at least one elastic element are selected such that for a cyclic motion the force exerted varies by less than 50% for at least 66% of the cycle.

19. The wearable assistive device according to claim 1, wherein the elasticities of the first force transmitting interconnection arrangement and of the at least one elastic element are selected such that for a cyclic motion the force exerted varies by less than 50% for at least 75% of the cycle.

20. The wearable assistive device according to claim 1, wherein the elasticities of the first force transmitting interconnection arrangement and of the at least one elastic element are selected such that for a cyclic motion the force exerted varies by less than 50% for at least 90% of the cycle.
